(19) United States
(12) Patent Application Publication
(10) Pub. No.: US 2026/0369399 A1
(43) Pub. Date: Nov. 19, 2026

(54) MEDICAL DEVICE REMOTE SCREEN VIEW METHODS, APPARATUS, AND SYSTEM

(71) Applicant: GAMBRO LUNDIA AB, Lund (SE)

(72) Inventors: Derek Neiman, Bethel, MN (US); Matthew Bivans, Naples, FL (US); Krishnan Lalu, Bangalore (IN); Velumani Pradeepkumar, Bangalore (IN)

(21) Appl. No.: 19/143,807

(22) PCT Filed: Dec. 20, 2023
(86) PCT No.: PCT/EP2023/087100

§ 371 Date: Jun. 26, 2025

Related U.S. Application Data
(60) Provisional application No. 63/435,439, filed on Dec. 27, 2022.

(51) Int. Cl. G16H 40/67; G06F 3/04847; G06V 10/75; G06V 20/62; G06V 30/10

(52) U.S. Cl. CPC G16H 40/67; G06F 3/04847; G06V 10/751; G06V 20/62; G06V 30/10

(57) ABSTRACT

Medical device remote screen view methods, apparatuses, and systems are disclosed. The systems, methods, and apparatuses are configured to provide a graphical representation of a screen that is currently displayed by a medical device to provide enough context as though a clinician were physically located at a patient's bedside. In some embodiments, the medical device transmits medical data and an identifier of a displayed screen, which is populated into a corresponding user interface of a software application on a clinician device. In other embodiments, the medical device transmits images of a displayed screen, which are displayed by the software application on a clinician device.

### BACKGROUND

Due to various causes, a person's renal system can fail. Renal failure produces several physiological derangements. For instance, it is no longer possible for a person with renal failure to balance water and minerals or to excrete daily metabolic load. Additionally, toxic end products of metabolism, such as, urea, creatinine, uric acid, and others may accumulate in a patient's blood and tissue.

Reduced kidney function and, above all, kidney failure is treated with dialysis. Dialysis removes waste, toxins, and excess water from a patient's body that normal functioning kidneys would otherwise remove. Dialysis treatment for replacement of kidney functions is critical to many people because the treatment is lifesaving.

One type of kidney failure therapy is Hemodialysis (“HD”), which in general uses diffusion to remove waste products from a patient's blood. A diffusive gradient occurs across a semi-permeable dialyzer between the blood and an electrolyte solution, called dialysate or dialysis fluid, to cause diffusion. The diffusion occurs externally from the patient, where an extracorporeal circuit is used for removing uncleansed blood and returning cleansed blood to the patient.

Hemofiltration (“HF”) is an alternative renal replacement therapy that relies on a convective transport of toxins from a patient's blood. HF is accomplished by adding substitution or replacement fluid to the extracorporeal circuit during treatment. The substitution fluid and the fluid accumulated by the patient in between treatments is ultrafiltered over the course of the HF treatment, providing a convective transport mechanism that is particularly beneficial in removing middle and large toxic molecules.

Hemodiafiltration (“HDF”) is a treatment modality that combines convective and diffusive clearances. HDF uses dialysis fluid flowing through a dialyzer, similar to standard hemodialysis, to provide diffusive clearance. In addition, substitution solution is provided directly to the extracorporeal circuit, providing convective clearance.

Another type of kidney failure therapy is peritoneal dialysis (“PD”), which infuses a dialysis solution, also called dialysis fluid, into a patient's peritoneal chamber via a catheter. The dialysis fluid is in contact with the peritoneal membrane in the patient's peritoneal chamber. Waste, toxins, and excess water pass from the patient's bloodstream, through the capillaries in the peritoneal membrane, and into the dialysis fluid due to diffusion and osmosis, i.e., an osmotic gradient occurs across the membrane. An osmotic agent in the PD dialysis fluid provides the osmotic gradient. Used or spent dialysis fluid is drained from the patient, removing waste, toxins, and excess water from the patient. This cycle is repeated multiple times.

There are various types of peritoneal dialysis therapies, including continuous ambulatory peritoneal dialysis (“CAPD”), automated peritoneal dialysis (“APD”), tidal flow dialysis, and continuous flow peritoneal dialysis (“CFPD”). CAPD is a manual dialysis treatment. Here, the patient manually connects an implanted catheter to a drain to allow used or spent dialysis fluid to drain from the peritoneal chamber. The patient then switches fluid communication so that the patient catheter communicates with a bag of fresh dialysis fluid to infuse the fresh dialysis fluid through the catheter and into the patient. The patient disconnects the catheter from the fresh dialysis fluid bag and allows the dialysis fluid to dwell within the peritoneal chamber, where the transfer of waste, toxins, and excess water takes place. After a dwell period, the patient repeats the manual dialysis procedure, for example, four times per day. Manual peritoneal dialysis requires a significant amount of time and effort from the patient, leaving ample room for improvement.

Automated peritoneal dialysis (“APD”) is similar to CAPD in that the dialysis treatment includes drain, fill and dwell cycles. APD machines, however, perform the cycles automatically, typically while the patient sleeps. APD machines free patients from having to manually perform the treatment cycles and from having to transport supplies during the day. APD machines connect fluidly to an implanted catheter, to a source or bag of fresh dialysis fluid and to a fluid drain. APD machines pump fresh dialysis fluid from a dialysis fluid source, through the catheter and into the patient's peritoneal chamber. APD machines also allow for the dialysis fluid to dwell within the chamber and for the transfer of waste, toxins, and excess water to take place. The source may include multiple liters of dialysis fluid including several solution bags.

APD machines pump used or spent dialysate from the patient's peritoneal cavity, though the catheter, and to the drain. As with the manual process, several drain, fill, and dwell cycles occur during dialysis. A “last fill” may occur at the end of the APD treatment. The last fill fluid may remain in the peritoneal chamber of the patient until the start of the next treatment, or may be manually emptied at some point during the day.

In any of the above modalities, the automated dialysis machine is located in a medical center or a patient's home. During a treatment, a clinician may need to review treatment progress, treatment setup, or an alert generated by an automated dialysis machine. For treatments that occur in a patient's home, it is extremely difficult for a clinician to travel to the automated machine. Instead of traveling, a clinician has to call the patient to help assess the alert or setup issue. Oftentimes, the clinician has to rely on a description provided by the patient, which may not be accurate or include enough information or context. Alternatively, the clinician may use a device to access the patient's electronic medical record (“EMR”), which includes a data log of treatment data and events generated by the automated dialysis machine. However, since the data is in a timestamped log form comprising strings of text and numbers, it can be difficult for a clinician to quickly diagnose the issue. Further, there are concerns the data in the log may not be representative of the data shown on a screen of the automated dialysis machine, further complicating the issue. Even for automated dialysis machines located in a medical facility, it may not be efficient or possible for a clinician to visit the patient and the automated dialysis machine to help identify and resolve a treatment or setup issue. Moreover, even when a clinician can visit the automated dialysis machine, the clinician only has a current-live view of the screen and would have to sift through data logs to review the history of the dialysis setup or treatment, which is tedious and time consuming.

A need accordingly exists for a dialysis system that provides clinicians additional context regarding a dialysis treatment without the clinician needing to be present at an automated dialysis machine.

### SUMMARY

Example systems, methods, and apparatus are disclosed herein that provide a remote view of a user interface of an automated dialysis machine. The systems, methods, and apparatus are configured to provide a graphical representation of a screen that is currently displayed by an automated dialysis machine to provide enough context as though the clinician were physically located at a patient's bedside. As described herein, there are a number of different ways the systems, methods, and apparatus can provide a remote view of a screen of an automated dialysis machine.

In some embodiments, the systems, methods, and apparatus only transmit medical data, a screen identifier, and a timestamp from an automated dialysis machine. In these embodiments, a software application operating on a clinician device stores un-populated user interface templates of the possible screens that can be displayed by the automated dialysis machine. The software application receives the medical data, the screen identifier, and the timestamp, and uses the screen identifier to select the corresponding un-populated user interface. The software application then uses labels provided with the medical data to populate corresponding data fields within the user interface to generate a populated user interface. Such a configuration enables the software application to display the same screen that is displayed by the automated dialysis machine without having to transmit a screen image, which consumes moderate amounts of bandwidth and processing.

The use of the timestamps enables the software application to create a screen progression video to show how the automated dialysis machine performed overtime as new medical data is generated, thereby providing additional context to help a clinician diagnose a potential setup or treatment issue. The video-feature enables a clinician to view what was happening at the automated dialysis machine before the issue occurred, which may be useful in determining the root cause of the issue. In addition to storing the screen progression video within the software application, the systems, methods, and apparatus disclosed herein may additionally or alternatively store the screen progression video to a memory device of the automated dialysis machine, to a database managed by a medical server, and/or a cloud-based database.

In some instances, bandwidth is further reduced by the automated dialysis machine only transmitting the medical data when at least some of the medical data changes. The automated dialysis machine may only transmit the medical data on a current displayed screen that has changed or transmit all the medical data shown on the screen when at least some of the data changes. Further, some of the user interfaces (and screens) may include scales, graphs, gauges, or other graphical elements that change based on a value of the related medical data. The user interfaces include coding that accordingly changes the graphical elements based on the related value of the medical device data.

In alternative embodiments, the systems, methods, and apparatus are configured to generate an image, such a JPEG image, a PNG image, etc. of a screen at the automated dialysis machine. The systems, methods, and apparatus are configured to provide the image to the software application. Each image may be timestamped, enabling the images to be sequenced to create a screen progression video. In these alternative embodiments, a virtual network computing (“VNC”) server receives and stores the images to a shared memory or cloud-based database, which may be protected with an address of the automated dialysis machine and a password. The software application enables a user to provide the address of the automated dialysis machine and the password to access the images. While these alternative embodiments may use more network bandwidth to transmit an image of a screen of the automated dialysis machine, the alternative embodiments require less processing related to selecting and populating user interfaces with data for display on the software application.

In some embodiments, the systems, methods, and apparatus are configured to provide confirmation that the medical data that is represented as being shown on a screen of the automated dialysis machine is accurate. In these embodiments, the systems, methods, and apparatus are configured to include a camera that is directed to a screen of the medical device. Images recorded by the camera are transmitted to the server and/or the software application for display adjacent to the populated user interface and/or the screen image. A clinician may visually compare the camera image to the populated user interface and/or the screen image to ensure the medical data is accurate and current and to ensure the screen displayed is correct. In some instances, the software application and/or the server may perform optical character recognition and/or a pixel analysis between the camera image and the populated user interface and/or the screen image to provide an automated confirmation. When there is a mismatch, the software application and/or the server may generate an alert to indicate the populated user interface and/or the screen image may not be accurate.

In light of the disclosure herein and without limiting the disclosure in any way, in a first aspect of the present disclosure, which may be combined with any other aspect listed herein, a system for remotely viewing medical data displayed on a screen of a medical device includes a medical device for performing a medical treatment for a patient. The medical device includes a plurality of different screens that may be shown on a display device, each screen being assigned an identifier and configured to show a subset of medical data related to the medical treatment. The medical device is configured to transmit an identifier of a screen that is displayed by the display device of the medical device and medical data that is shown on the screen. The system also includes a user device including a processor and a memory, the memory storing instructions, which when executed by the processor, cause the processor to operate a software application, which includes a plurality of un-populated user interfaces that correspond to the plurality of different screens of the medical device. Each user interface includes at least one data field corresponding to the subset of medical data that is associated with the related screen. The system further includes a server communicatively coupled to the medical device and the user device via a network. The server is configured to receive the identifier of the screen that is displayed by the medical device and the medical data that is shown on the screen. After receiving a request message from the software application of the user device, the server is configured to transmit the identifier of the screen that is displayed by the medical device and the medical data that is shown in the screen, causing the software application to select an un-populated user interface that corresponds to the identifier of the screen and populate the at least one data field of the selected user interface with the received medical data.

In a second aspect of the present disclosure, which may be combined with any other aspect listed herein, the medical device is further configured to transmit a timestamp with the identifier of the screen and the medical data that is shown on the screen, and the server is further configured to store, to a medical record associated with the patient, the timestamp in association with the identifier of the screen that is displayed by the medical device and the medical data that is shown on the screen to a medical record associated with the patient.

In a third aspect of the present disclosure, which may be combined with any other aspect listed herein, the server is further configured to receive a second timestamp, subsequent to the first timestamp, and at least one of (i) a second identifier of a second screen that is displayed by the display device of the medical device and second medical data that is shown on the second screen, or (ii) the identifier of the screen that is displayed by the display device of the medical device and updated medical data that is shown on the screen. The server additionally stores the at least one of (i) or (ii) to the medical record associated with the patient.

In a fourth aspect of the present disclosure, which may be combined with any other aspect listed herein, the software application is further configured to for (i) select a second un-populated user interface that corresponds to the second identifier of the screen, populate the at least one data field of the selected second user interface with the received second medical data, and create a screen progression video using the user interface associated with the timestamp and the second user interface associated with the second timestamp.

In a fifth aspect of the present disclosure, which may be combined with any other aspect listed herein, the software application is further configured to for (ii) update the at least one data field of the selected user interface with the received updated medical data, and create a screen progression video using the user interface associated with the timestamp and the user interface associated with the second timestamp.

In a sixth aspect of the present disclosure, which may be combined with any other aspect listed herein, the software application includes an option to play the screen progression video, fast forward the screen progression video, rewind the screen progression video, slow a play rate of the screen progression video, or skip to a next alarm or event provided within the screen progression video.

In a seventh aspect of the present disclosure, which may be combined with any other aspect listed herein, the software application is further configured to transmit the request message to the server after the medical treatment is finished.

In an eighth aspect of the present disclosure, which may be combined with any other aspect listed herein, the medical data includes data labels, and the software application is configured to populate the at least one data field of the selected user interface with the received medical data by matching the data labels of the medical data to the at least one data field of the selected user interface.

In a ninth aspect of the present disclosure, which may be combined with any other aspect listed herein, the server is configured to store the identifier of the screen that is displayed by the medical device and the medical data that is shown on the screen to a medical record associated with the patient, the medical record including an identifier of the patient, an identifier of the medical device, and medical treatment session information. The request message includes at least one of the identifier of the patient, the identifier of the medical device, or the medical treatment session information. The request message causes the server to select the identifier of the screen that is displayed by the medical device and the medical data that is shown in the screen for transmission to the software application.

In a tenth aspect of the present disclosure, which may be combined with any other aspect listed herein, the medical device includes at least one of a hemodialysis machine, a hemodiafiltration machine, a large-volume infusion pump, a syringe pump, a patient-controlled analgesia pump, a parenteral nutrition pump, a peritoneal dialysis machine, a continuous renal replacement therapy (“CRRT”) machine, a ventilator, a physiological monitor/sensor, or a patient bedside monitor.

In an eleventh aspect of the present disclosure, which may be combined with any other aspect listed herein, the server is a cloud-based server or provided within a medical facility.

In a twelfth aspect of the present disclosure, which may be combined with any other aspect listed herein, the medical data includes at least one of an ultrafiltration (“UF”) volume, a treatment time, a concentrate type, a sodium concentration, a bicarbonate concentration, a heparin bolus volume, a heparin flow rate, a heparin stop time, a heart rate, a dialysis fluid temperature, a dialysis fluid flow rate, dialysis fluid conductivity, UF profiling data, sodium profiling data, bicarbonate profiling data, a blood flow rate to a dialyzer, pre-and post-infusion volumes, and an indication as to whether UF is isolated, an indication whether a single needle is being used for arterial and venous connections, an event, an alarm, or diagnostic information.

In a thirteenth aspect of the present disclosure, which may be combined with any other aspect listed herein, at least one of the un-populated user interfaces is an overlay user interface that is displayed over another user interface.

In a fourteenth aspect of the present disclosure, which may be combined with any other aspect listed herein, the medical device is configured to receive physiological data from at least one sensor, and include the physiological data with the medical data.

In a fifteenth aspect of the present disclosure, which may be combined with any other aspect listed herein, the server is configured to receive physiological data from at least one sensor, and include the physiological data with the medical data.

In a sixteenth aspect of the present disclosure, which may be combined with any other aspect listed herein, a system for remotely viewing medical data displayed on a screen of a medical device includes a user device including a processor and a memory, the memory storing instructions, which when executed by the processor, cause the processor to operate a software application, which includes a plurality of un-populated user interfaces that correspond to a plurality of different screens of a medical device. Each user interface includes at least one data field corresponding to a subset of medical data that is associated with the related screen. The system also includes a server communicatively coupled to a medical device and the user device via a network. The server is configured to receive, from the medical device, identifiers of screens that are displayed by the medical device, the medical data that is shown on each screen in relation to a medical treatment for a patient, and timestamps when the medical data was displayed, generated, or transmitted. After receiving a request message from the software application of the user device, the server is configured to transmit the identifiers of the screens, the corresponding medical data, and the related timestamps, causing the software application to select un-populated user interfaces that correspond to the identifiers of the screens, populate the at least one data field of the selected user interfaces with the received medical data, arrange the user-interfaces into a sequence based on the timestamps, and cause the user-interfaces to be shown in a video-like sequential order to replay the medical treatment.

In a seventeenth aspect of the present disclosure, which may be combined with any other aspect listed herein, the software application is configured to transmit the request message to the server after the medical treatment is finished.

In an eighteenth aspect of the present disclosure, which may be combined with any other aspect listed herein, the server is configured to store the identifiers of the screens, the medical data, and the corresponding timestamps to a medical record including at least one of an identifier of the patient, an identifier of the medical device, and medical treatment session information. The request message includes at least one of the identifier of the patient, the identifier of the medical device, or the medical treatment session information. The request message causes the server to select the identifiers of the screens, the medical data, and the corresponding timestamps for transmission to the software application.

In a nineteenth aspect of the present disclosure, which may be combined with any other aspect listed herein, a system for remotely viewing a screen of a medical device includes a medical device for performing a medical treatment for a patient includes a display device configured to display a medical treatment screen including medical data, a processor configured to record an image of the medical treatment screen and compress the image, and a digital communication module configured to receive the compressed image, decompress the image, and transmit the image to a server for storage in a memory device. The system also includes a user device including a processor and a memory, the memory storing instructions, which when executed by the processor, cause the processor to operate a software application. The system further includes a server communicatively coupled to the medical device and the user device via a network. The server is configured to receive the image from the digital communication module, and after receiving a request message from the software application of the user device, transmit the image, causing the software application to display the image.

In a twentieth aspect of the present disclosure, which may be combined with any other aspect listed herein, the processor of the medical device is configured to receive a remote view input and record the image of the medical treatment screen after receiving the remote view input.

In a twenty-first aspect of the present disclosure, which may be combined with any other aspect listed herein, the processor is configured to cause at least one of an IP address or a password to be displayed after the remote view input is received, and transmit the at least one of the IP address or the password to the server for assessing the image. The software application is configured to display a prompt for the at least one of the IP address or the password to access the image associated with the medical device.

In a twenty-second aspect of the present disclosure, which may be combined with any other aspect listed herein, the processor of the medical device is configured to associate a timestamp with the recorded image.

In a twenty-third aspect of the present disclosure, which may be combined with any other aspect listed herein, the processor associates the timestamp with the recorded image by at least one of storing the timestamp as metadata for the image, displaying a watermark of the timestamp on the image, or storing the timestamp in conjunction with the image within a memory of the medical device.

In a twenty-fourth aspect of the present disclosure, which may be combined with any other aspect listed herein, the processor is further configured to record next images of the medical treatment screen every one second to thirty seconds, associate respective timestamps with the next images, and compress the next images. The digital communication module is configured to decompress the next images and transmit the next images to the server. Further, the server transmits the next images to the software application causing the software application to order the next images based on the respective timestamps and display the ordered next images.

In a twenty-fifth aspect of the present disclosure, which may be combined with any other aspect listed herein, the processor is further configured to record next images of the medical treatment screen when a new medical treatment screen is displayed or at least some of the medical data changes, associate respective timestamps with the next images, and compress the next images. The digital communication module is configured to decompress the next images and transmit the next images to the server. Further, the server transmits the next images to the software application causing the software application to order the next images based on the respective timestamps and display the ordered next images.

In a twenty-sixth aspect of the present disclosure, which may be combined with any other aspect listed herein, the server is a virtual network computing (“VNC”) server and the software application is a VNC viewer application.

In a twenty-seventh aspect of the present disclosure, which may be combined with any other aspect listed herein, the system further includes a camera configured to record a camera image of the medical treatment screen, and transmit the camera image to the server. The server is configured to transmit the camera image to the software application causing the software application to display the camera image in conjunction with the image of the medical treatment screen created by the medical device.

In a twenty-eighth aspect of the present disclosure, which may be combined with any other aspect listed herein, at least one of the server or the software application is configured to compare the camera image to the image of the medical treatment screen created by the medical device, and when the camera image does not match the image of the medical treatment screen created by the medical device, provide an auditory and/or visual indication that the image of the medical treatment screen created by the medical device is not correct.

In a twenty-ninth aspect of the present disclosure, which may be combined with any other aspect listed herein, at least one of the server or the software application is configured to perform the comparison using optical character recognition or a pixel analysis.

In a thirtieth aspect of the present disclosure, which may be combined with any other aspect listed herein, the medical device is configured to show a barcode on the medical treatment screen and the camera image is configured to include the barcode. The at least one of the server or the software application is configured to perform the comparison by comparing the barcode in the image recorded by the medical device to the barcode included within the camera image.

In a thirty-first aspect of the present disclosure, which may be combined with any other aspect listed herein, the medical device is configured to periodically update the barcode.

In a thirty-second aspect of the present disclosure, any of the structure and functionality disclosed in connection with FIGS. 1 to 12 may be combined with any of the other structure and functionality disclosed in connection with FIGS. 1 to 12.

In light of the present disclosure and the above aspects, it is therefore an advantage of the present disclosure to provide a remote view of a screen of a medical device.

It is another advantage of the present disclosure to transmit only a screen identifier and medical data shown on the screen of a medical device to enable a software application of a clinician device to populate a corresponding user interface.

It is further advantage of the present disclosure to transmit recorded images of a medical device screen for viewing in a software application of a clinician device.

Additional features and advantages are described in, and will be apparent from, the following Detailed Description and the Figures. The features and advantages described herein are not all-inclusive and, in particular, many additional features and advantages will be apparent to one of ordinary skill in the art in view of the figures and description. Also, any particular embodiment does not have to have all of the advantages listed herein and it is expressly contemplated to claim individual advantageous embodiments separately. Moreover, it should be noted that the language used in the specification has been selected principally for readability and instructional purposes, and not to limit the scope of the inventive subject matter.

### BRIEF DESCRIPTION OF THE FIGURES

FIG. 1 shows a diagram of a medical system with a cloud-based server, according to an example embodiment of the present disclosure.

FIG. 2 shows a diagram of a medical system with a medical center-based server, according to an example embodiment of the present disclosure.

FIG. 3 is a diagram that shows how user interfaces may be indexed on a memory device to enable a screen of a medical device to be viewed remotely, according to an example embodiment of the present disclosure.

FIG. 4 is a diagram of a user interface displayed by a software application on a clinician device, according to an example embodiment of the present disclosure.

FIG. 5 is a diagram of another user interface displayed by the software application of the clinician device, according to an example embodiment of the present disclosure.

FIG. 6 is a diagram of another user interface that may be displayed by the software application operating on the clinician device, according to an example embodiment of the present disclosure.

FIG. 7 shows flow diagrams of example procedures for providing a remote view replay of a treatment session using medical data, according to an example embodiment of the present disclosure.

FIG. 8 is a flow diagram of an example procedure for providing a remote view replay of a treatment session using images, according to an example embodiment of the present disclosure.

FIG. 9 is a diagram of a medical system that uses a VNC for a remote screen view of a medical device, according to an example embodiment of the present disclosure.

FIGS. 10 and 11 are diagrams of a medical device (e.g., an automated dialysis machine) for providing screens for remote viewing, according to an example embodiment of the present disclosure.

FIG. 12 is a diagram of a medical system with a camera to provide verification of the remote screen view, according to an example embodiment of the present disclosure.

### DETAILED DESCRIPTION

Methods, systems, and apparatus are disclosed for remote screen viewing of a medical device. The methods, systems, and apparatus are configured to provide a real time, near-real time, and/or replayable view of a medical device at a remote clinician device. Such a configuration enables a clinician to see how a medical device is currently operating or previously operated to help diagnose a setup or treatment issue for a patient. The methods, systems, and apparatus provide an almost exact reproduction of what is displayed on the medical device to provide complex context for the medical data.

Clinicians are accustomed to working directed with medical devices and are familiar with the different screens. This familiarity enables clinicians to quickly diagnose issues at a medical device. The example methods, systems, and apparatus provide a clinician with the same information in the same context and graphical format while the clinician is located remote from the medical device, thereby enabling the clinician to assess a treatment issue as though the clinician were present at the patient's bedside. Such a feature helps improve patient care for home-based dialysis and in remote regions where one clinician may be responsible for overseeing many different medical facilities. The remote view feature disclosed herein overcomes issues of known medical systems that simply display logs of medical device data, which are difficult to understand and identify trends.

The remote view feature described herein may also help clinicians with less training or experience. Usually, when a clinician that is new to a medical device begins a setup or treatment and experiences and issue, it is common for the clinician to contact a service desk to report an issue with the device. It can generally take ten to thirty minutes to resolve the issue. With the remote view feature disclosed herein, the service desk can obtain a real time or near-real time view of a screen of the medical device to help guide the clinician through the setup procedure or treatment. The service desk can view how the clinician changes the screen of the medical device and provide appropriate feedback.

The remote view feature also alleviates clinicians from having to enter a patient's room. This may be especially important for patients with infectious diseases such as Covid where a clinician would normally have to don protective masks and clothing to enter the patient's room. Instead of going into a patient's room, the remote view feature enables clinicians to have the same view of a medical device screen shown on their own mobile device or computer.

In addition to above, the methods, systems, and apparatus are configured to create a screen progression video using a sequence of images or medical data from a medical device. The screen progression video enables a clinician to review how a medical device operated in the past before an issue was detected, thereby helping the clinician to determine possible root causes. For example, using the screen progression video, a clinician may notice that a patient's blood pressure slowly started to elevate when heparin or another fluid was infused into an extracorporeal circuit of an HD machine. Since the blood pressure rise was slow, a time duration of an hour may have elapsed between the blood pressure reaching a threshold for triggering an alert. Known medical devices only show a current status of a treatment. In contrast to known medical devices, the methods, systems, and apparatus enable a clinician to rewind a screen progression video to see what happened at a medical device when the patient's blood pressure began to rise. As described in more detail below, the screen progression video may be stored on the medical device, at a database, and/or on a software application operating on a clinician device. The remote view feature provided by the methods, systems, and apparatus enable a clinician to view a near-exact copy of a medical device screen from virtually any location, thereby improving patient care when a clinician cannot easily reach a patient's bedside.

Reference is made herein to automated HD or HDF machines. It should be appreciated that the methods, systems, and apparatus may provide remote screen viewing for any type of medical device including a large-volume infusion pump, a syringe pump, a patient-controlled analgesia pump, a parenteral nutrition pump, a PD machine, a CRRT machine, a ventilator, a physiological monitor/sensor, and/or a patient bedside monitor. Each of these medical devices includes a defined number of screens that enables a software application on a clinician device to populate a corresponding user interface with the relevant medical data. Further, each of these medical devices may be configured to record and transmit a screen image over a network for display at a clinician device.

Reference is made herein to medical data. As disclosed, medical data is generated at a medical device and is available for transmission. The medical data includes treatment programming information. Treatment programming information includes one or more parameters that define how a medical device is to operate to administer a treatment to a patient. For a peritoneal dialysis therapy, the parameters may specify an amount (or rate) of fresh dialysis fluid to be pumped into a peritoneal cavity of a patient, an amount of time the fluid is to remain in the patient's peritoneal cavity (i.e., a dwell time), and an amount (or rate) of used dialysis fluid and ultrafiltration (“UF”) that is to be pumped or drained from the patient after the dwell period expires. For a treatment with multiple cycles, the parameters may specify the fill, dwell, and drain amounts for each cycle and the total number of cycles to be performed during the course of a treatment (where one treatment is provided per day or separate treatments are provided during the daytime and during nighttime). For CRRT, the parameter may include a continuous UF rate. In addition, the parameters may specify dates/times/days (e.g., a schedule) in which treatments are to be administered by the medical fluid delivery machine. Further, parameters of a prescribed therapy may specify a total volume of dialysis fluid to be administered for each treatment in addition to a concentration level of the dialysis fluid, such as a dextrose level. For an infusion therapy, the parameters may include a volume to be infused, a medication to be infused, a medication concentration, a medication dosage, and/or an infusion rate.

For the AK 98™ hemodialysis machine manufactured by Baxter International Inc., the treatment programming parameters may include a UF volume, a treatment time, a concentrate type, a sodium concentration, a bicarbonate concentration, a heparin bolus volume, a heparin flow rate, a heparin stop time, a heart rate monitoring flag, a dialysis fluid temperature, a dialysis fluid flow rate, and indications as to whether UF is isolated, UF profiling is to occur, a single needle is being used for arterial and venous connections, whether sodium profiling is to occur, whether bicarbonate profiling is to occur, and/or whether dialysis fluid conductivity monitoring is to occur. A blood flow rate to a dialyzer may also be a treatment programming parameter. For HDF, pre-and post-infusion volumes may also be treatment programming parameters.

The medical data also includes event information that relates to administration of the treatment. The event information may include data generated by a medical device that is indicative of measured, detected, or determined parameter values. For example, while a prescribed therapy may specify that a treatment is to comprise five separate cycles, each with a 45 minute dwell time, a medical fluid delivery device may administer a treatment where fewer cycles are provided, each with a 30 minute dwell time. The medical device monitors how the treatment is administered and accordingly provides parameters that are indicative of the operation. The parameters for the treatment data may include, for example, a total amount of dialysis fluid administered to the patient, a number of cycles operated, a fill amount per cycle, a dwell time per cycle, a drain time/amount per cycle, an estimated amount of UF removed, a treatment start time/date, and/or a treatment end time/date. The treatment data may also include calculated parameters, such as a fill rate and a drain rate, determined by dividing the amount of fluid pumped by the time spent pumping. The treatment/event data may further include an identification of an alarm that occurred during a treatment, a duration of the alarm, a time of the alarm, an event associated with the alarm, and/or an indication as to whether the issue that caused the alarm was resolved or whether the alarm was silenced.

The medical data further includes device machine logs that include diagnostic information, fault information, etc. The diagnostic information may include information indicative of internal operations of a medical device, such as faults related to pump operation, signal errors, communication errors, software issues, etc. The diagnostic information may also include setup information, such as steps performed to connect tubing to the medical device and prime/disinfect the tubing. The medical data may be transmitted as a data stream or provided at periodic intervals. In some instances, the medical data may be transmitted as events or other changes to the data occur.

The methods, systems, and apparatus are described within the context of providing a remote view of a screen of a medical device. In some embodiments, the remote view feature may be provided in conjunction with the remote control of a medical device. For instance, an application on a clinician device may display the screen of the medical device as disclosed herein. The application may also provide inputs in relation to the displayed screen that enable a clinician to change an operation of the medical device. In some instances, the inputs may resemble or be identical to the inputs as displayed by the screen of the medical device. Selection of an input causes a corresponding command message to be transmitted to the medical device to provide remote control.

### I. Medical Data Remote View Embodiment

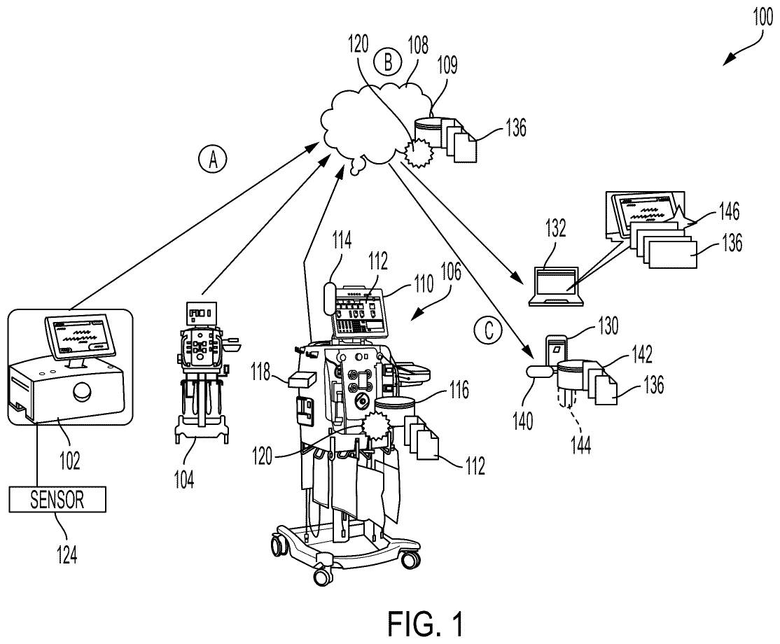

FIG. 1 shows a diagram of a medical system 100, according to an example embodiment of the present disclosure. The example medical network system 100 includes three medical devices 102, 104, and 106 (e.g., automated dialysis machines). The automated dialysis machine 102 may be the Amia Automated PD system manufactured by Baxter® International Inc. The automated dialysis machines 104 and 106 may be he PrisMax CRRT machine manufactured by Baxter International Inc. In other embodiments, the system 100 may include additional medical devices or fewer medical devices. For example, the system 100 may include an intermediate hemodialysis machine, an infusion pump (e.g., a syringe pump, a linear peristaltic pump, a large volume pump (“LVP”), an ambulatory pump, multi-channel pump), a nutritional compounding machine, a water preparation machine, etc. The system 100 may also include a bedside patient monitor or physiological sensors such as an oxygen sensor, a respiratory monitor, a glucose meter, a blood pressure monitor, an electrocardiogram (“ECG”) monitor, a weight scale, a heart rate monitor, or any other peripheral medical device configured to sense a physiological parameter of a patient.

The automated dialysis machines 102 to 106 may be located in a patient's home or within a medical facility. For example, the automated dialysis machine 102 may be located within a patient's home while the automated dialysis machines 104 and 106 are located in a medical facility. FIG. 1 shows the automated dialysis machines 102 to 106 communicatively coupled to a server 108 via one or more networks. The server 108 may be a cloud-based system that includes one or more distributed memory devices 109.

The automated dialysis machines 102 to 106 are configured to accept one or more parameters specifying a treatment or prescription (i.e., treatment programming information). During operation, the automated dialysis machines 102 to 106 generate event, diagnostic, and/or operational data (e.g., medical data). In some embodiments, the medical data conforms to the ISO/IEEE 11073™ standards as XML-based information objects. In other embodiments, the medical data is in a different format, such as JavaScript Object Notation (“JSON”), a HyperText Markup Language (“HTML”), a comma-separated values (“CSV”), text, and/or Health-Level-7 (“HL7”).

For brevity, only components of the automated dialysis machine 106 are described below. However, it should be appreciated that the same or similar components may be included with the automated dialysis machines 102 and 104. Such components are independent of the type of medical device.

In the illustrated example, the automated dialysis machine 106 includes a display device 110 that shows one or more screens 112 providing a graphical representation of medical data, and more generally a status of a patient treatment or setup. The display device 110 may include a touchscreen that is configured to receive inputs. Additionally or alternatively, the display device 110 may include control interface 114 that enable a clinician to select options shown on the screen 112. The control interface 114 may include buttons, a control panel, or the touchscreen of the display device 110. The control interface 114 may also be configured to enable a clinician to navigate to a certain screen 112 for display. The control interface 114 may also provide instructions for operating or controlling the automated dialysis machine 106.

The automated dialysis machine 106 also includes a memory device 116 that is configured to store the screens 112. The automated dialysis machine 106 has a present number of screens and popup windows that may be displayed. For example, the automated dialysis machine 106 may have twenty or thirty different screens 112 for treatment setup, alerts, and types of treatments. Further, in some embodiments, multiple screens are displayed where at least some screens may be overlaid on other screens. When multiple screens are used, the automated dialysis machine 106 may identify the screen stack and/or positioning to enable user interfaces provided at clinician devices to be arranged in a similar manner. Each screen 112 is assigned an identifier. Further, each screen includes data fields that specify which of the medical data generated by the automated dialysis machine 106 is to be displayed within a certain section of the screen 112. The data fields may specify a data label or other identifier that is used for matching and determining to which data field labeled medical data is to be populated. The memory device 116 may include any RAM, ROM, EEPROM, flash drive, solid state drive, distributed database, etc.

The automated dialysis machine 106 further includes a processor 118 that is communicatively coupled to the display device 110, the control interface 114, and the memory device 116. The processor 118 is configured to generate and/or process medical data 120, which is stored in the memory device 116. The processor 118 determines which subset of the medical data 120 is to be displayed on the screen 112 of the automated dialysis machine 106. The processor 118 may generate and process the medical data 120 in a HL7 format, an XML format, a binary version 2 format, a binary version 3 format, or a Fast Healthcare Interoperability Resources (“FHIR”) format.

As discussed above, the medical data 120 for the automated dialysis machine 106 may include a UF volume, a treatment time, a concentrate type, a sodium concentration, a bicarbonate concentration, a heparin bolus volume, a heparin flow rate, a heparin stop time, a heart rate monitoring flag, a dialysis fluid temperature, a dialysis fluid flow rate, and indications as to whether UF is isolated, UF profiling is to occur, a single needle is being used for arterial and venous connections, whether sodium profiling is to occur, whether bicarbonate profiling is to occur, and/or whether dialysis fluid conductivity monitoring is to occur. The medical data 120 may also include a blood flow rate to a dialyzer and pre-and post-infusion volumes. Further, the medical data 120 may include events and/or alarms, such as priming information, line connection/disconnection information, disinfection/cleaning information, occlusion detections, significant pressure or flow rate fluctuations or changes, etc. The processor 118 creates medical data 120 in conjunction with operating one or more pumps or other components to administer the treatment. The medical data 120 may further include a prescription value for a total treatment time during which a patient is to be submitted to a blood treatment, a prescription value for a total patient fluid removal to be achieved by an end of a total treatment time, and a prescription value for an average patient fluid removal rate to be kept across the total treatment time.

In some embodiments, one or more physiological sensors may be communicatively coupled to one of the automatic dialysis machines 102 to 106. For instance, FIG. 1 shows a physiological sensor 124 connected to the home-based automatic dialysis machine 102. The sensor 124 may include an oxygen sensor, a respiratory monitor, a glucose meter, a blood pressure monitor, an electrocardiogram (“ECG”) monitor, a weight scale, a heart rate monitor, etc. The processor 118 of the automated dialysis machines 102 to 106 may be configured to include data from the physiological sensors as the medical data 120 that is transmitted to the server 108 since some of the screens 112 may include data fields for patient physiological data measured by one or more sensors.

The processor 118 of the automated dialysis machine 106 operates according to one or more instructions for performing a treatment on a patient. The instructions may be acquired via the control interface 114 or via the memory device 116. The processor 118 also monitors devices components for issues, which are documented as diagnostic medical data 120. While the automated dialysis machine 106 is shown as having one processor 118, as discussed below, in some embodiments, the automated dialysis machine 106 may include two or more processors 118 with specified operations.

The automated dialysis machines 102 to 106 are communicatively coupled to the server 108 via one or more network connections. For the home-based automated dialysis machine 102, the network connection may include any combination of an Ethernet connection, an Internet connection, Wi-Fi connection, a wireless local area network (“WLAN”), and/or a cellular 5G/6G connection. For the facility-based automated dialysis machines 104 and 106, the network connection may include any combination of an Ethernet connection, a Wi-Fi connection, a WLAN connection, a LAN connection, etc. The network connections may include one or more of an access point, a router, repeater, or other telecommunication equipment for routing communications in a network.

In the illustrated example, the server 108 is configured to store the medical data 120 from the automated dialysis machines 102 to 106. The medical data 120 may be transmitted periodically or every time at least some of the medical data 120 changes. The server 108 stores the medical data 120 to the memory device 109 to enable remote access by clinician devices 130 and 132. Such a configuration prevents the clinician devices 130 and 132 from directly accessing the automated dialysis machines 102 to 106. In some embodiments, the memory device 109 may also store un-populated user interfaces 136 that correspond to the different screens 112 of the automated dialysis machines 102 to 106.

FIG. 1 shows an example where the server 108 is provisioned in a cloud-based network. To transmit the medical data 120, the automated dialysis machines 102 to 106 may authenticate with the server 108 and transmit using secure Internet communication protocols. The automated dialysis machines 102 to 106 may be configured to transmit the medical data 120 to one or more destination addresses that correspond to one or more application programming interfaces (“APIs”) of the server 108. Together, the server 108 and the memory device 109 may include a network of servers and memory devices that are configured in a distributed framework. In some instances, the server 108 and the memory device 109 may be hosted by Amazon® Web Services (“AWS”).

The server 108 is configured to store the medical data 120 to the memory device 109 by patient identifier and/or machine identifier/address. For instance, messages with the medical data 120 from the automated dialysis machines 102 to 106 may include a patient identifier and/or a machine identifier/address. The server 108 uses the identifier/address for indexing and storing the medical data 120 to the appropriate location within the memory device 109, which enables the clinician devices 130 and 132 to access the medical data 120 by providing the corresponding patient or machine identifier after authenticating.

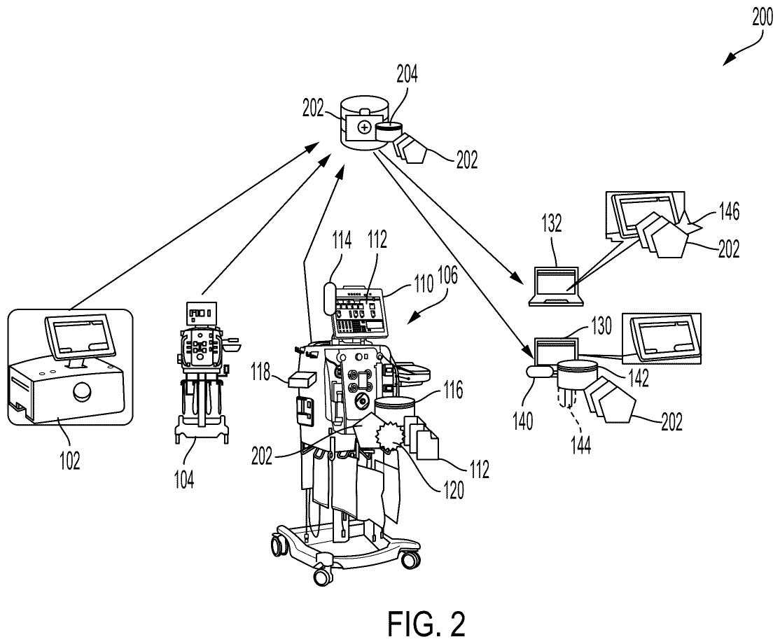

In contrast to FIG. 1, FIG. 2 shows a medical system 200 that includes a server 201 that may be located in a medical facility. The medical system 200 also includes a memory device 204 that is also centrally located with respect to the server 201. In some instances, the server 201 may include a VNC server.

In the example of FIG. 2, the automated dialysis machines 104 and 106 may be connected to the server 201 via one or more gateways, routers, and/or switches that form part of a LAN. The home-based automated dialysis machine 102 instead connects to the server 201 through a firewall to the LAN. In either embodiment, the medical systems 100 and 200 of FIGS. 1 and 2 respectively enable medical data 120 to be transmitted from the automated dialysis machines 102 to 106 to a server for storage on a memory device regardless of a network location of the server and memory device.

Returning to FIG. 1, the medical system 100 includes the clinician devices 130 and 132 for accessing the medical data 120 stored at the memory device 109. The clinician devices 130 and 132 may include smartphones, tablet computers, laptop computers, desktop computers, workstations, clinician stations, etc. While FIG. 1 shows two clinician devices 130 and 132, it should be appreciated that the medical system 100 may include a plurality of clinician devices that are connected to the server 108 via a local or wide area network.

For brevity, only the clinician device 130 is described. However, the forgoing disclosure of the clinician device 130 also applies to the other clinician devices 132. The clinician device 130 includes a processor 140 and a memory device 142. Instructions are stored on the memory device 142. Execution of the instructions by the processor 140 causes the processor 140 to operate a software application 144 to perform the operations described herein. The processor 140 may comprise digital and/or analog circuity structured as a microprocessor, application specific integrated circuit (“ASIC”), controller, etc. The memory device 142 includes a volatile or non-volatile storage medium. Further, the memory device 142 may include a solid state or disk storage medium.

The software application 144 is configured to store un-populated user interfaces 136 to the memory device 142. As described below, the local storage of the user interfaces 136 enables the software application 144 to select, populate, and display a user interface 136 after receiving a screen identifier and corresponding medical data 120 from the server 108. In other embodiments, the server 108 transmits the corresponding user interface 136 when the medical data 120 is transmitted to the software application 144 on the clinician device 130 such that local storage of the user interfaces is not needed.

To provide a remote view of the medical data 120, the automated dialysis machines 102 to 106 transmit generated or created medical data 120 to the server 108, as shown at Event A. The medical data 120 includes a screen identifier of the screen 112 that is displayed by the display device 110 of the automated dialysis machine 106, for example. The medical data 120 also includes medical data 120 that is shown within the screen 112 (which is a subset of all the generated medical data), as determined by the processor 118. The medical data 120 can include prescription information, flow rates, pressures, alarm identifiers, etc. The medical data 120 may also include a timestamp corresponding to when the medical data 120 is generated or displayed within the screen 112. The medical data 120 may further include key press information regarding actuation of the control interface 114 and/or screen overlay information (e.g., an alarm dialog box placed on top of a therapy screen). The medical data 120 may also include a patient or machine identifier.

The automated dialysis machine 106 may be configured to transmit the medical data 120 periodically, such as every one second, every two seconds, every thirty seconds, etc. Alternatively, the processor 118 of the automated dialysis machine 106 is configured to transmit only the medical data 120 shown on the screen 112 when such data changes. In yet alternative embodiments, the automated dialysis machine 106 transmits all of the medical data 120 shown on the screen 112 anytime at least some of the data changes. As a default, the processor 118 may be configured to transmit the medical data 120 every thirty minutes, for example, when there are changes to the medical data values shown on a currently displayed screen 112. It should be appreciated that regardless of which transmission method used, the size of the information transmitted is only a few bytes.

At Event B, the server 108 stores the medical data 120 based on the patient or machine identifier. The server 108 may store the medical data 120 to an EMR of the patient, which is stored in a data structure or database of the memory device 109. The server 108 is configured to continually store newly received medical data 120 such that prior medical data 120 is not overwritten.

At Event C in FIG. 1, the software application 144 of the clinician device 130 first authenticates with the server 108. After authentication, the software application 144 transmits a request message to the server 108. The request message includes, for example, a patient or machine identifier to enable the server 108 to locate the corresponding medical data 120 stored in the memory device 109. The request message may instead specify a therapy session and/or date. In some embodiments, the software application 144 operates with the server 108 to provide a selectable and filterable list of patients, treatment sessions, and/or medical devices for a given patient, treatment day, care area, etc.

The request message causes the server 108 to identify and transmit the requested medical data 108 to the software application 144. The server 108 also transmits the corresponding screen identifier, timestamp, key press information, screen overlay information, and parameters that are part of the medical data 120. The software application 144 uses the screen identifier to select the corresponding un-populated user interface 136 that is stored in the memory device 142. Each un-populated user interface 136 is provided in an indexed array within the memory device 142 based on identifier, which enables the software application 144 to quickly determine which user interface is needed based on a received screen identifier. The software application 144 next populates the medical data 120 into data fields of the selected user interface 136 to provide a live or near-live view of the screen 112 of the automated dialysis machine 106.

In some embodiments, the screen identifier may include a version number or the screen identifier may correspond to a version number. When a new version of a screen is created, the server 108 and/or the software application 144 may also receive an updated corresponding user interface 136. The incorporation of a version number ensures that the appropriate user interface 136 is selected regardless of software updates and releases.

Further, in some embodiments, the screen identifier may include or take into consideration a language. For example, a screen identifier may include a prefix or suffix that specifies a language. This ensures a user interface 136 of the appropriate language is selected.

When multiple screens are shown together and/or overlaid on each other, multiple screen identifiers are transmitted. The automated dialysis machine 106 may include a screen layer and/or screen position location within a message with the screen identifier. Such information is used by the server 108 and/or the software application 144 to select, arrange, and position corresponding user interfaces 136.

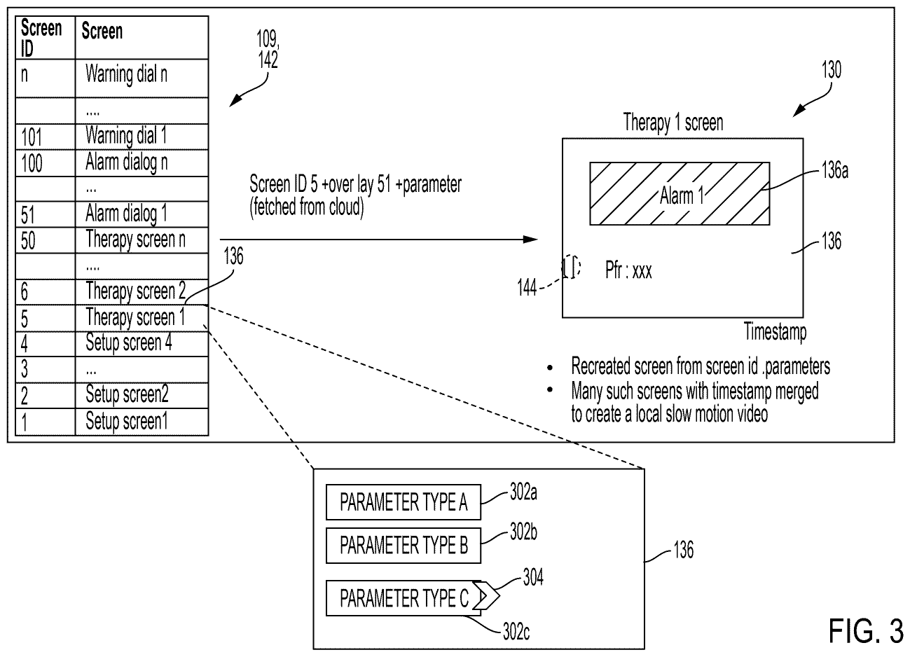

FIG. 3 is a diagram that shows how the user interfaces 136 may be indexed on the memory devices 109 and 142, according to an example embodiment of the present disclosure. In the illustrated example, each un-populated user interface 136 includes a screen identifier and a corresponding name. Each un-populated user interface 136 includes one or more data fields 302 that identify which medical data 120 is to be written thereto. In some embodiments, the medical data 120 includes data labels or identifiers that specify a type of each data value. In these embodiments, the software application 144 matches the identifier of the data field 302 to the data label of the medical data 120 to determine which of the medical data 120 is to be written to that data field. In other embodiments, an order at which the medical data 120 is received specifies the data type, which is used for matching to the appropriate data field 302.

Each of the data fields 302 may specify a location on the user interface 136 to where the medical data 120 is to be displayed. The data fields 302 may further specify font size, font type, font color, and any other information for appropriately rendering the medical data 120 such that it matches or approximates how the medical data is shown on the screen 112 of the automated dialysis device 106, for example.

As shown in FIG. 3, the data fields 302a and 302b may be single value fields that specify a single numerical value is to be displayed. In contrast, the data field 302c includes one or more rules 304 that specify how a graphical element within the user interface 136 is to be adjusted based on a value of the corresponding medical data 120. The rules 304 may be used for gauges, graphs, meters, or other variable graphical elements of a user interface 136.

In the illustrated example, the software application 144 determines that Screen ID 5 is to be rendered. Additionally, the software application 144 determines that overlay screen 51 is to be displayed. The software application 144 uses the medical data 144 received or otherwise fetched from the server 108 to display Alarm Dialog user interface 136a overlaid on the Therapy 1 user interface 136 to match what is shown on the display device 110 of the automated dialysis machine 106.

Key presses of the control interface 114 are not shown on the screen 112 of the automated dialysis device 106. However, the software application 144 may provide a graphical representation of the control interface 114 and highlight when a button is pressed or provide an audio indication. The graphical representation of the control interface 114 may be provided adjacent to the displayed user interface 136.

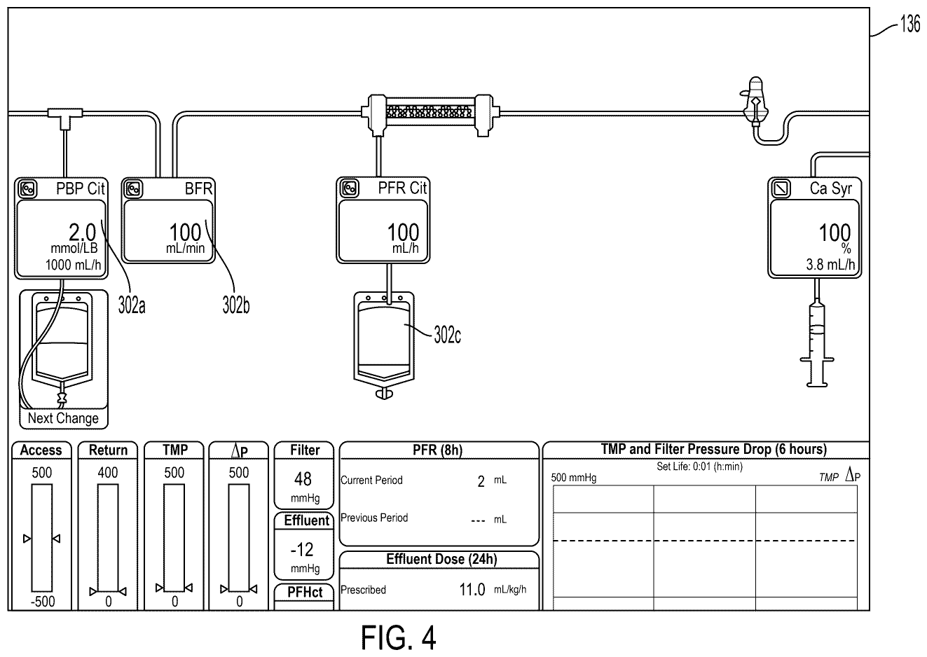

FIG. 4 is a diagram of the Therapy 1 user interface 136 displayed by the software application 144 on the clinician device 130, according to an example embodiment of the present disclosure. The illustrated user interface 136 is representative of the screen 112 shown on the automated dialysis machine 106. The user interface 136 includes a plurality of data fields 302, such as data fields 302a and 302b, that show respective an infusion rate and a blood pump rate or blood flow rate. The un-populated user interface includes the graphics while the software application 144 adds the values for the medical data 120. The data field 302c includes a value displayed in addition to a graphical element that can be varied based on the value of the medical data. Specifically, the graphic shows a patient fluid removal volume corresponding to a fluid removal rate. As the volume increases, the rule 304 for the data field 302 specifies that container graphical element is to appear fuller. The rule 304 may include a correlation between the removal volume and the displayed fluid height.

The user interface 136 of FIG. 4 includes other data fields including single value displays, graphs, gauges, etc. The software application 144 populates each of the data fields and adjusts the variable graphics such that the populated user interface appears almost identical to the screen 112 of the automatic dialysis machine 106.

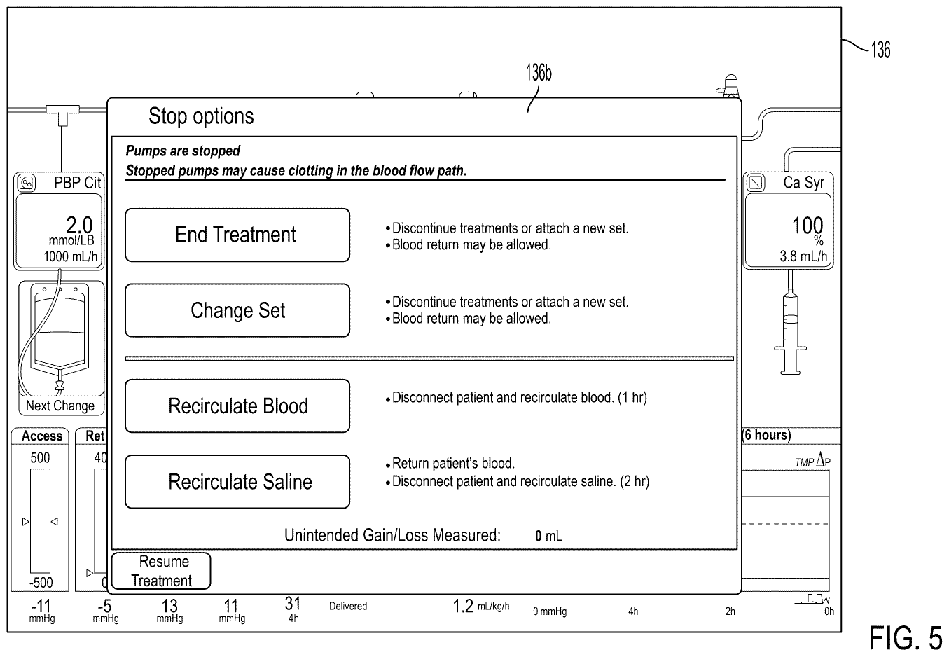

FIG. 5 is a diagram of another user interface 136b displayed by the software application 144 of the clinician device 130, according to an example embodiment of the present disclosure. The user interface 136b is an overlay displayed on top of the user interface 136 displayed in connection with FIG. 4. At this junction, a user may have pressed a button of the control interface 114 to pause or stop a treatment, which causes an overlay screen to be displayed by the automatic dialysis machine 106. The identifier of the overlay screen is transmitted from the automatic dialysis machine 106 to the sever 108, which is stored to the appropriate EMR within the memory device 109. In addition, an indication of which button was pressed, a timestamp of the action, and any corresponding medical data 120 is also transmitted. The software application 144 determines the overlay screen is displayed based on the received screen identifier, and accordingly selects the corresponding overlay user interface 136b. It should be appreciated that while a user at the automated dialysis machine 106 may select any of the options of the overlay screen, the clinician using the software application 144 can only view the user interface 136b without being able to select the options.

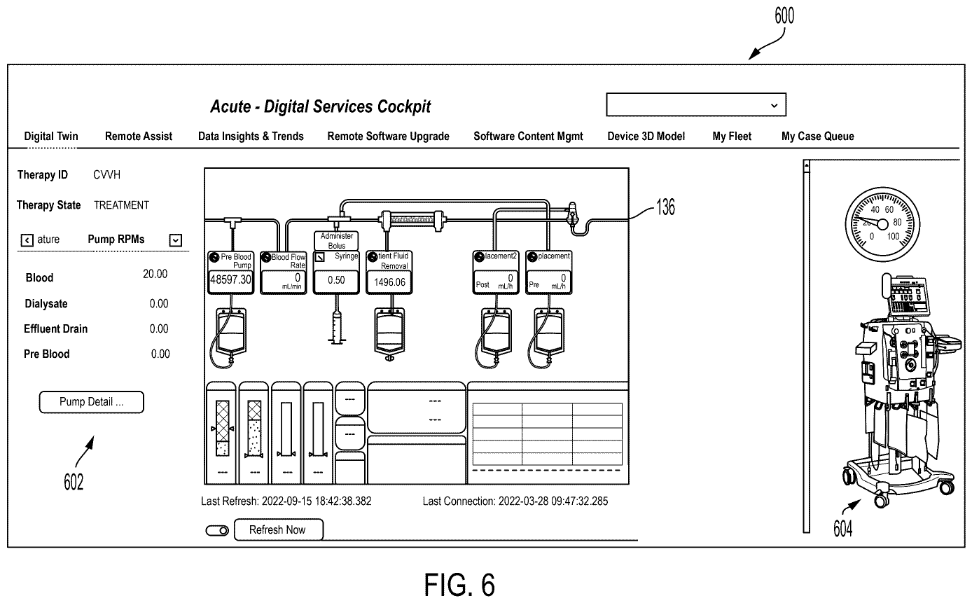

FIG. 6 is another user interface 600 that may be displayed by the software application 144 operating on the clinician device 130, according to an example embodiment of the present disclosure. Here, the user interface 600 includes a section for displaying the user interface 136 representing the screen 112 of the automated dialysis machine 106. Additionally, the user interface 600 includes section 602 for viewing a summary of the medical data 120, which may be organized into separate categories. The user interface 600 also includes a section 604 that shows a graphical illustration of the automated dialysis machine 106 to provide some additional context for a clinician.

The above-description provides for a current-view of the screen 112 of the automated dialysis machine 106. As new medical data 120 is generated and stored to the memory device 109, the software application 144 fetches the new data 120 to update the user interface 136. Further, as new screens 112 are displayed on the display device 11, the software application 144 receives new screen identifiers from the server 108 for rendering the appropriate user interface 136 In addition to current views, the software application 144 and the server 108 use the timestamps to create a screen progression video from a sequence of the displayed user interfaces 136. Since each instance of the medical data 120 is timestamped, a separate instance of the user interface 136 can be created. The sequences of the user interfaces 136 can be combined into a video-type playback that enables a clinician to pause, rewind, fast-forward, etc. using the software application 144 to view a setup or treatment progression.

When a clinician selects to view a treatment or setup already in progress, the software application 144 and/or the server 108 is configured to construct a screen progression video 146 as the user interfaces 136 are updated overtime. Further, the software application 144 and/or the server 108 may be configured to obtain the medical data 120 (and corresponding timestamps, screen identifiers, etc.) that was generated before the clinician made the remote view request. As such, the software application 144 and/or the server 108 backfills the setup or treatment procedure to enable a clinician to rewind to view the entire progress up to a current point.

Returning to FIG. 1, the clinician device 132 may request to view a treatment that has already ended. The clinician device 132 transmits a request message with a treatment session identifier, patient identifier, and/or machine identifier. In response, the server 108 fetches the medical data 120 (including timestamps, screen identifiers, button presses, etc.) that are associated with the requested session. The server 108 transmits the medical data 120 to the software application on the clinician device 132, which then populates user interfaces 136 accordingly to generate a screen progression video 146. In some embodiments, the video 146 is a sequence of images of the user interfaces 136 as they change overtime. In other embodiments, the video 146 is a sequence of the user interfaces 136 themselves for each timestamp of medical data 120. Regardless, the screen progression video 146 enables a clinician to review how a dialysis setup or treatment progressed.

In some instances, the server 108 may create the screen progression video 146 instead of the software application 144. In these instances, the server 108 creates the screen progression video 146 after receiving the request message with the treatment session, patient, and/or machine selection. The server 108 then transmits the screen progression video 146 for display on the clinician device 132, for example.

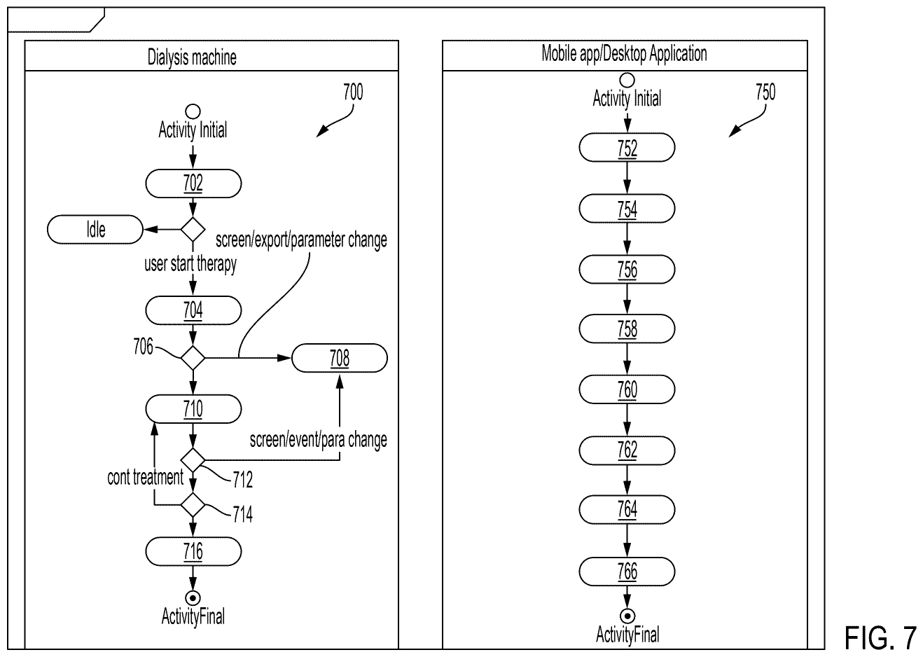

FIG. 7 shows flow diagrams of example procedures 700 and 750 for providing a remote view replay of a treatment session, according to an example embodiment of the present disclosure. Although the procedures 700 and 750 are described with reference to the flow diagrams illustrated in FIG. 7, it should be appreciated that many other methods of performing the steps associated with the procedures 700 and 750 may be used. For example, the order of many of the blocks may be changed, certain blocks may be combined with other blocks, and many of the blocks described may be optional. For example, a clinician may instead select to view a current setup or treatment rather than replaying an already finished procedure. The operations described in the procedure 700 are specified by one or more instructions and may be performed among multiple devices including, for example, the server 108 and/or the automated dialysis machines 102 to 106. The operations described in the procedure 750 are specified by one or more instructions of the software application 144 and may be performed among multiple devices including, for example, the server 108 and/or the clinician device 130.

The example procedure 700 begins when the automated dialysis machine 106 initializes or starts up (block 702). The automated dialysis machine 106 idles until a clinician or other user starts a therapy setup (block 704). The automated dialysis machine 106 then determines if new medial data 120 has been generated (block 706). When new medical data 120 is generated, the automated dialysis machine 106 transmits the newly generated medical data 120 including a screen identifier, dialysis parameters, timestamp, button presses, patient identifier, etc. to the server 108 for storage in the appropriate patient EMR of the memory device 109 (block 708). After setup is complete and a therapy or treatment is started (block 710), the automated dialysis machine 106 determines if new medial data 120 has been generated (block 712). When new medical data 120 is generated, the automated dialysis machine 106 transmits the newly generated medical data 120 including a screen identifier, dialysis parameters, timestamp, button presses, patient identifier, etc. to the server 108 for storage in the appropriate patient EMR of the memory device 109 (block 708). The automated dialysis machine 106 then checks if the treatment is to continue (block 714). If the treatment is to continue, the automated dialysis machine 106 continues sending newly generated medical data 120 to the server 108 until the treatment is ended (block 716). The example procedure 700 then ends.

The example procedure 750 begins when the software application 144 is opened or activated (block 752). The software application 144 may display a list of available automated dialysis machines and/or other medical devices (block 754). The software application 144 may enable a user to enter a dialysis machine identifier (e.g., a serial number), a treatment date and/or duration (e.g., a start date and an end date), and/or a patient identifier (blocks 756 and 758). The software application 144 uses the filter criteria or a selection of a patient, medical device, treatment session, etc. to transmit a request message to the server 108 (block 760). The request message is used to fetch the medical data 120 from the memory device 109 via the server 108 (block 762). This includes the screen identifiers, timestamps, button presses, etc.

The software application 144 then creates a screen progression video 146 by, for each timestamp, populating a user interface 136 corresponding to the related screen identifier with the associated medical data 120 (block 764). The software application 144 then arranges the user interfaces 136 in an order based on the timestamps to create the screen progression video 146. In some instances, the software application 144 may also record a video of a progression through the different interfaces to create a native video file for viewing the therapy progression.

The clinician then views the screen progression video 146, which may include options for pausing, playing, fast forwarding, rewinding, and changing a playing speed (block 766). In some instances, the screen progression video 146 has an option to play until an event occurs, where then the screen progression video 146 is paused. The software application 144 may also include a scroll bar that a clinician may drag to reach a certain point in the treatment. In some instances, icons or other graphical elements are displayed in conjunction with the scroll bar to indicate the temporal location of events/alarms/alerts during the treatment. Below is a summary of the possible video modes provided by the software application 144:

### a) Auto Run-Pause-Run (Pause on Events)

In this mode review video will play automatically. Video will stop automatically at every event (alarm, warning, error, screen change etc.). Pause for few seconds and then run again.

Timestamp will be displayed as a watermark on top of video or on the scrollbar.

### b) Manual Run-Pause-Run (Pause on Events)

Video will start playing and pause on event (alarm, warning, error, screen change etc.). However, user needs to tap on a screen for video to continue to play. (Gives the user time to review a screen).

Timestamp will be displayed as a watermark on top of video or on the scrollbar.

### c) Selected Events Snapshots:

In this option a user can select one or multiple events (e.g., access pressure high or blood leak alarm).

In this mode video has only selected events screens with a timestamp on scroll bar or as watermark.

The video stops at each event and tap on the screen to continue.

### d) Selected Phase Snapshot (Show Only Setup or Therapy Screens or Recirculation etc.):

In this option, a user can select if they want to see only therapy screens or setup screens.

### e) Fast Forward and Stop at Selected Event.

In this option, video will play as a fast forward video but stops at events selected by a user.

After the clinician has finished viewing the screen progression video 146, the example procedure 750 ends.

### II. Screen Image Remote View Embodiment

In the above-embodiments, the software application 144 is configured to populate user interfaces with medical data. In the embodiments discussed below, the medical device is configured to transmit an image of the screen displayed. While these embodiments may use more bandwidth to transmit images instead of medical data, these embodiments use less processing since the medical data does not have to be populated into one or more user interfaces by a software application operating on a clinician device. Similar to the earlier embodiments, the medical device may transmit timestamps with the screen images to enable a progression video of the screens to be created, thereby enabling a replay of a medical treatment, such as a dialysis treatment.

Returning to FIG. 2, the medical system 200 shows the automated dialysis machine 106 generating images 202 of the screen 112 shown by the display device 110. The images 202 may be recorded using a screen capture operation. Each image 202 may be a JPEG, a PNG, a BMP, a TIFF, or other image file format. The automated dialysis machine 106 may generate the image 202 periodically, such as every second, every two seconds, every ten seconds, every thirty seconds, every five minutes, etc. Alternatively, the automated dialysis machine 106 is configured to create the image 202 when at least some of the medical data 120 on the screen 112 changes or a different screen is shown. The automated dialysis machine 106 also creates a timestamp when the image 202 is generated. The timestamp may be stored to the image 202 (e.g., the image file) as metadata, written over the image as a watermark, or stored in association with the image 202 within the memory device 116.

As shown in FIG. 2, after the image 202 is generated, the automated dialysis machine 106 transmits the image 202 (and the corresponding timestamp) to the server 108 for storage to a patient's electronic medical record within the memory device 109. The image 202 may be stored to a record, a file, or a data structure that identifies the patient, treatment session, and/or the automated dialysis machine 106 to enable searching by the software application 144. In some instances, the automated dialysis machine 106 compresses the image 202, which is transmitted to the server 108 for decompression and storage. In other embodiments, the images 202 are transmitted uncompressed.

In addition to a timestamp, the automated dialysis machine 106 may also store information indicative of alarms or events in association with the image 202. The inclusion of alarm or event information enables the software application 144 to fast forward through a progression or sequence of images 202 until an alarm or event is reached.

In the illustrated example, the server 108 is shown as being located within a medical facility such that the automated dialysis machine 106 and the server 108 are located both behind a firewall as part of a local area network. In these embodiments, the image 202 may not be encrypted. Further, the automated dialysis machine 106 may not need to authenticate with the server 108 since both are part of a trusted network. Similar to the timestamp, the alarm or event information may be stored as metadata to the image 202, overlaid as a watermark, or stored in associated with the image 202.

In the example of FIG. 1, the server 108 may be located offsite, such as in a cloud-computing environment. In these embodiments, the automated dialysis machine 106 may need to authenticate with the server 108 before transmitting the image 202. Additionally or alternatively, the automated dialysis machine 106 may encrypt or otherwise secure the image 202 for transmission.

After the server 108 has stored at least one image 202, the software application 144 on the clinician device 130 may request to view the at least one image 202. As shown in FIG. 2, the images 202 may be viewed as a treatment is ongoing or after the treatment has ended. For instance, the clinician device 130 displays images 202 during an ongoing treatment such that new images 202 are received periodically. The software application 202 may compile each new image 202 into the screen progression video 146, which builds the video as images 202 are received and the treatment continues. Additionally, the clinician device 132 displays images 202 as a screen progression video 146 created after the treatment has ended. It should be appreciated that since an image of the screen 112 of the automated dialysis machine 106 is captured, any overlay screens are also captured in the same image 202.

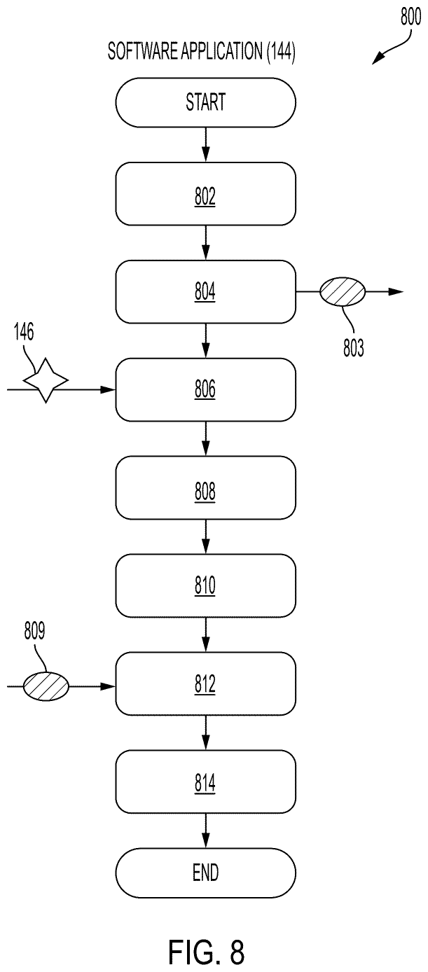

FIG. 8 is a flow diagram of an example procedure 800 for providing a remote view replay of a treatment session using images, according to an example embodiment of the present disclosure. Although the procedure 800 is described with reference to the flow diagram illustrated in FIG. 8, it should be appreciated that many other methods of performing the steps associated with the procedure 800 may be used. For example, the order of many of the blocks may be changed, certain blocks may be combined with other blocks, and many of the blocks described may be optional. For example, a clinician may instead select to view a current setup or treatment rather than replaying an already finished procedure. The operations described in the procedure 800 are specified by one or more instructions and may be performed among multiple devices including, for example, the server 108 and/or the clinician device 130.

The example procedure 800 begins by the software application 144 enabling a clinician to search and/or filter through indexed records managed by the server 108 to identify a desired dialysis treatment/setup (block 802). The software application 144 may enable the clinician to search/filter by patient identifier, medical device identifier, treatment session information (e.g., date, duration, time, etc.), etc. The software application then enables a clinician to select a treatment session, which causes a request message 803 to be transmitted to the server 108 (block 804). In some embodiments, the server 108 is configured to arrange and compile the images 202 associated with the selected treatment session into a screen progression video 146. Since each image 202 is associated with the timestamp, the server 108 is configured to order or sequence the images 202 in a chronological order. The server 108 then transmits the screen progression video 146 to the software application 144 (block 806).

In some instances, the software application 144 instead creates the screen progression video 146. In these instances, the server 108 transmits the images 202. The software application 144 then arranges the images 202 by timestamps to create a video sequence. The software application 144 next compiles the screen progression video 146 (block 808).

In some embodiments, the server 108 and/or the software application 144 is configured to reduce a size of the screen progression video 146 by only adding images 202 that are different from previous screens. The server 108 and/or the software application 144 may use a pixel compare operation to identify differences. Alternatively, the automated dialysis machine 106 only creates images 202 when the medical data 120 changes such that the server 108 and/or software application 144 do not need to look for and omit duplicate images 202.

As shown in FIG. 8, after the screen progression video 146 is compiled, the software application 144 displays the screen progression video 146 (block 810). The software application 144 may then receive one or more inputs 809 for playback control (block 812). The inputs 809 may include a control to play, play at a reduced speed, rewind, fast forward, stop, or fast forward to an alarm or event (that was stored in associated with the image 202). The software application 144 accordingly adjusts the screen progression video 146 based on the received playback control (block 814). The example procedure 800 continues until the clinician ends playback of the screen progression video 146.

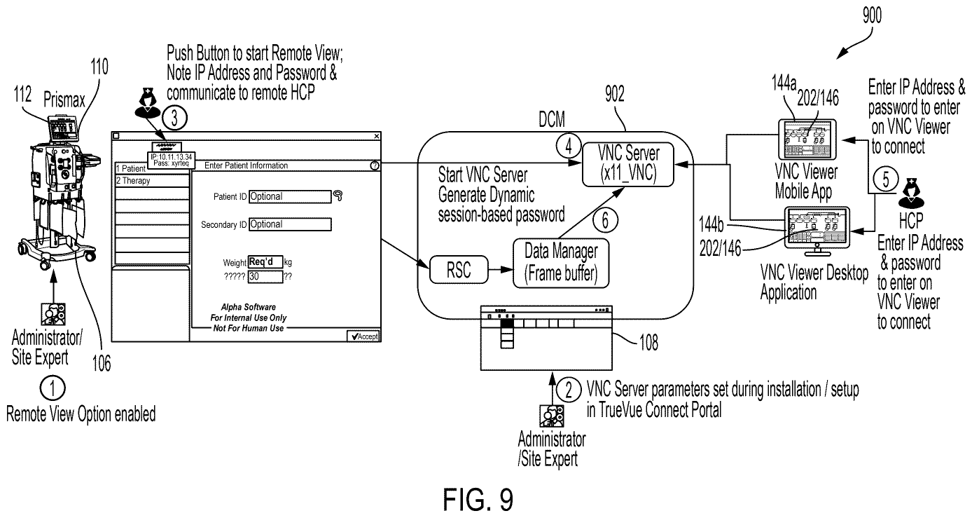

In some embodiments, the access to the images 202 and/or the screen progression playback video 146 may be safeguarded using a VNC framework. FIG. 9 is a diagram of a medical system 900 that uses a VNC for a remote screen view of a medical device, according to an example embodiment of the present disclosure. In the illustrated example, at Event 1, a service technician or site expert enables a remote view feature at the automated dialysis machine 106. At Event 2, the service technician or site expert then sets VNC parameters to enable the automated dialysis machine to connect to the server 108 (e.g., a VNC server in this embodiment). Next at Event 3, a clinician has to select a ‘Remote View’ button at the automated dialysis machine 106. Selecting the ‘Remote View’ button causes the automated dialysis machine 106 to record and transmit images 202 of the screen 112. As such, this embodiment only provides for remote view of the automated dialysis machine 106 when the option is selected at the machine itself.

Also at Event 3, the automated dialysis machine 106 is configured to display a network address (e.g., an IP address) and a password. At Event 4, the automated dialysis machine 106 transmits a message to the server 108 to indicate that the ‘Remote View’ feature was selected. The message may include the password. The message causes the server 108 to start a VNC session for the automated dialysis machine 106. The server 108 uses the password for access to the VNC session. At this point (Event 6), the automated dialysis machine 106 begins transmitting images 202 of the screen 112 to the server 108, which stores the images 202 in the memory device 109 in a data structure or file that is indexed to the VNC session, a patient, and/or the automated dialysis machine 106. The automated dialysis machine 106 may generate images of the screen 112 every one to third seconds, for example. Additionally or alternatively, the automated dialysis machine 106 may record an image of the screen 112 when at least some of the medical data changes and/or when a different screen is displayed by the display device 110.

In some embodiments, the automated dialysis machine 106 generates the image 202 of the screen 112, and then compresses the image for transmission to a digital communication module 902, which is a network interface for the automated dialysis machine 106. The digital communication module may then decompress the image as a frame buffer and then transmit the image to the server 108. In other examples, the image 202 is not compressed by the automated dialysis machine 106 or a compressed image 202 is transmitted to the server 108 from the digital communication module 902.

At Event 5, the software application 144 prompts a clinician for the network address and password. The software application 144 transmits the received network address and password to the server 108, which confirms the network address and password match the session password and network address of the automated dialysis machine 106. At this point, the software application 144 is authenticated by the server 108 to view the images 202 from the automated dialysis machine 108. The software application 144 receives the images 202 associated with the VNC session from the server 108. The software application 144 then displays the images 202. In some embodiments, the software application 144 or the server 108 arranges the images 202 into a chronological order based on corresponding timestamps to enable the images 202 to be shown as a screen progression video 146.

### III. Medical Device Data and Image Processing Embodiment

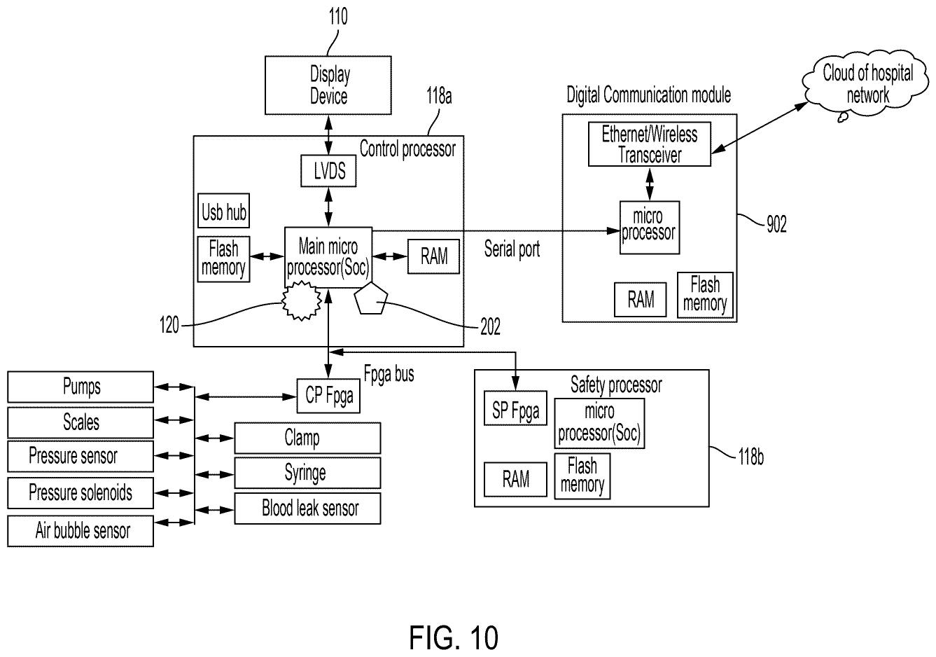

As discussed above, a remote view of a screen of a medical device may be displayed within a software application of a clinician device by transmitting medical data and a screen identifier or an image of the medical device screen. FIG. 10 is a diagram of the medical device (e.g., the automated dialysis machine 106) according to an example embodiment of the present disclosure. As shown in FIG. 10, a control processor 118a is communicatively coupled to the display device 110. The control processor 118a is also communicatively coupled to a safety processor 118b via a FPGA bus. The safety processor 118b is configured to provide fail safe operations for the automated dialysis machine 118a in the event the control processor 118a is not operable. The safety processor 118b may also handle start-up and shutdown operations.

The control processor 118a is also communicatively coupled to the digital communication module 902 via a serial or USB connection. The digital communication module 902 includes a transceiver, microprocessor, RAM, and flash memory to enable communication with a network. In some embodiments, the digital communication module 902 is included within the automated dialysis machine 106. In other embodiments, the digital communication module 902 is external and communicatively coupled to the automated dialysis machine 106.

The automated dialysis machine 106 also includes pumps, scales, pressure sensors, pressure solenoids, an air bubble sensor, clamps, a syringe pump, and a blood leak sensor, in some examples, that are connected to the control processor 118a via a CP FPGA. The pumps, scales, pressure sensors, pressure solenoids, an air bubble sensor, clamps, a syringe pump, and a blood leak sensor generate the medical data 120 discussed above. As shown in FIG. 10, the control processor 118a creates medical data 120 based on the operation of the pumps, scales, pressure sensors, pressure solenoids, an air bubble sensor, clamps, a syringe pump, and a blood leak sensor. The control processor 118a then incorporates the medical data 120 into one or more screens 112 that are displayed by the display device 110 via a LVDS. Further, in connection with some embodiments, the control processor 118a transmits the medical data 120 (and newly changed/generated medical data) to the server 108.

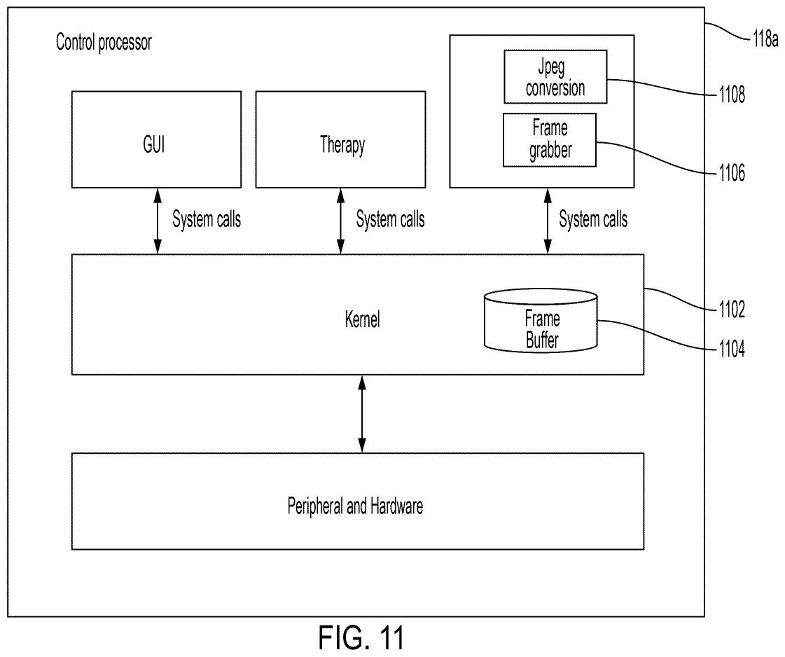

FIG. 11 is a diagram of the control processor 118a for processing the screens of the display device for the automated dialysis machine, according to an example embodiment of the present disclosure. The control processor 118a includes a kernel 1102 with a frame buffer 1104, which is a pixel buffer. The frame buffer 1104 is a continuous memory area that is located within the address space of the CPU of the control processor 118a. The frame buffer 1104 contains the current screen 112 that is shown on the display device 110. The content of the frame buffer 1104 is prepared by a graphics engine of the control processor 118a using software rendering or a graphics accelerator. For example, software rendering or a graphics accelerator is used to incorporate specified medical data 120 into a screen for display. The software rendering or graphics accelerator may use data values to change an appearance of some graphics and how those graphics are rendered. The kernel 1102 provides an option to read the frame buffer 1104 so that display applications can grab the frame buffer as needed for display on the display device 110.

To record an image 202 of the screen 112, a screen grabbing application 1106 operating on the control processor 118a reads the frame buffer 110 periodically (e.g., every second). The screen grabbing application 1106 then copies of the content of the frame buffer 1104 to an application buffer, which is then processed by a JPEG conversion application 1108 into a compressed JPEG image 202. The control processor 118a then transmits the compressed JPEG image 202 through a serial connection to the digital communication module 902, which decompresses the image 202.

### IV. Camera Verification Embodiment

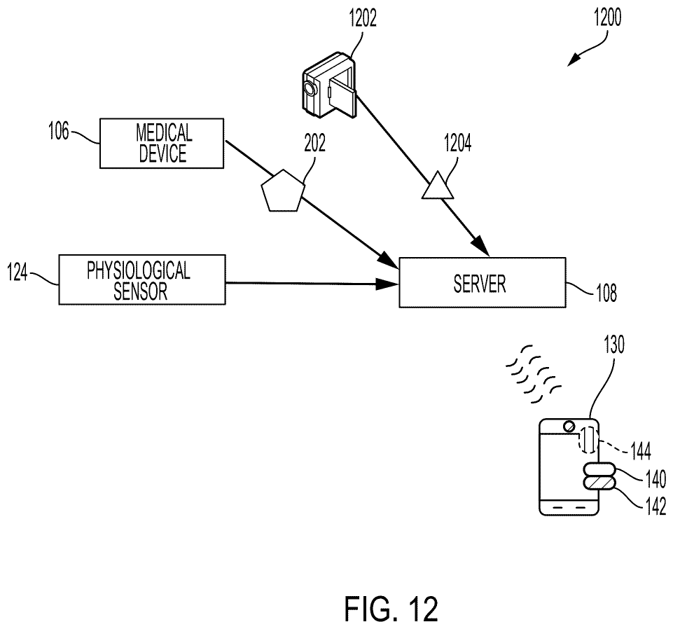

In some embodiments, the graphical representation of the screen of the medical device may be verified for accuracy. This verification provides a clinician assurance that currently viewed screen is correct. FIG. 12 is a diagram of a medical system 1200 with a camera 1202 to provide verification of the remote screen view, according to an example embodiment of the present disclosure. The camera 1202 is configured to record video or images 1204, which are transmitted to the server 108. The camera 1202 is positioned to face the display device 110 of a medical device (e.g., the automated dialysis machine 106) to record images of the screen 112.

The server 108 is configured to analyze the video or images 1204 to segment the screen 112. As discussed above, the medical device 106 also transmits the image 202 of the screen 112. The server 108 next compares the video or images 1204 from the camera 1202 to the image 202 from the frame buffer 1104 of the medical device 106. When there is a match, the server 108 may provide an indication that the image 202 is correct. When there is not a match, the server 108 may transmit an indication to the software application 144 to indicate the image 202 is not the current or most up to date screen view of the medical device 106. The indication may include an auditory and/or visual indication. In some embodiments, the server 108 may not perform the comparison and the software application 144 instead displays the video or images 1204 from the camera 1202 in conjunction with the images 202 to enable a clinician to make the comparison. In yet other embodiments, the software application 144 performs the comparison.

The server 108 and/or the software application 144 may perform the comparison using a pixel analysis. For example, the server 108 and/or the software application 144 may compare pixel color values of the video or images 1204 from the camera 1202 to pixel color values of the images 202. A favorable comparison is made if the pixels have approximately the same values in the same locations. In other embodiment, the server 108 and/or the software application 144 may perform optical character recognition to identify text within the images 1204 and 202. The server 108 and/or the software application 144 then compares the identified text to determine whether there is a match.

In some embodiments, the comparison may be made between the video or images 1204 from the camera 1202 and the medical data 120 populated into the user interface 136. The system 1200 would operate in the same manner as described above but instead of comparing images, the medical data and corresponding user interface is used for the comparison. For example, the server 108 and/or the software application 144 may perform an OCR routine on the video or images 1204 from the camera 1202 to identify the medical data 120. The server 108 and/or the software application 144 also receives the medical data 120 from the medical device 106 or the images 202. When the images 202 are received, the server 108 and/or the software application 144 perform an OCR routine to identify the medical data for comparison to the identified medical data from the camera images 1204. When the medical data 120 is received, the data itself is compared to the medical data from the camera images 1204. The comparison may be performed periodically (e.g., every minute, two minutes, five minutes, fifteen minutes, etc. or when updated medical data is detected or new images 202 or 1202 are received.

An output from the camera 1202 may be associated at the server 108 with the medical device 106. In other embodiments, the camera 1202 may be directly coupled to the clinician device 130 to enable the software application 144 to perform the comparison. In these examples, the images 1204 are not transmitted to the server 108 but instead are transmitted from the camera 1202 to the software application 144 via, for example, a wired or wireless connection.

In some embodiments, the medical device 106 may display a barcode or other code on the screen 112. The medical device 106 may periodically update the barcode, for example, every one second to thirty seconds. In these embodiments, the server 108 and/or the software application 144 is configured to compare the barcode in the images 1204 and 202 to determine a favorable comparison. The comparison may be made using a pixel analysis. The use of the camera 1202 accordingly ensures the remote screen view is accurate for a clinician.

### V. Conclusion

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

1. A system for remotely viewing medical data displayed on a screen of a medical device, the system comprising:
a medical device for performing a medical treatment for a patient, the medical device including a plurality of different screens that may be shown on a display device, each screen being assigned an identifier and configured to show a subset of medical data related to the medical treatment, the medical device configured to transmit
an identifier of a screen that is displayed by the display device of the medical device, and
medical data that is shown on the screen;
a user device including a processor and a memory, the memory storing instructions, which when executed by the processor, cause the processor to operate a software application, the software application including a plurality of un-populated user interfaces that correspond to the plurality of different screens of the medical device, each user interface including at least one data field corresponding to the subset of medical data that is associated with the related screen; and
a server communicatively coupled to the medical device and the user device via a network, the server configured to:
receive the identifier of the screen that is displayed by the medical device and the medical data that is shown on the screen, and
after receiving a request message from the software application of the user device, transmit the identifier of the screen that is displayed by the medical device and the medical data that is shown in the screen, causing the software application to
select an un-populated user interface that corresponds to the identifier of the screen, and
populate the at least one data field of the selected user interface with the received medical data.

2. The system of claim 1, wherein the medical device is further configured to transmit a timestamp with the identifier of the screen and the medical data that is shown on the screen, and wherein the server is further configured to store, to a medical record associated with the patient, the timestamp in association with the identifier of the screen that is displayed by the medical device and the medical data that is shown on the screen to a medical record associated with the patient.

3. The system of claim 2, wherein the server is further configured to:
receive a second timestamp, subsequent to the first timestamp, and at least one of:
(i) a second identifier of a second screen that is displayed by the display device of the medical device and second medical data that is shown on the second screen, or
(ii) the identifier of the screen that is displayed by the display device of the medical device and updated medical data that is shown on the screen; and additionally store the at least one of (i) or (ii) to the medical record associated with the patient.

4. The system of claim 3, wherein the software application is further configured to:
for (i) select a second un-populated user interface that corresponds to the second identifier of the screen;
populate the at least one data field of the selected second user interface with the received second medical data; and
create a screen progression video using the user interface associated with the timestamp and the second user interface associated with the second timestamp.

5. The system of claim 3, wherein the software application is further configured to:
for (ii) update the at least one data field of the selected user interface with the received updated medical data; and
create a screen progression video using the user interface associated with the timestamp and the user interface associated with the second timestamp.

6. The system of claim 4, wherein the software application includes an option to play the screen progression video, fast forward the screen progression video, rewind the screen progression video, slow a play rate of the screen progression video, or skip to a next alarm or event provided within the screen progression video.

7. The system of claim 1, wherein the software application is further configured to transmit the request message to the server after the medical treatment is finished.

8. The system of claim 1, wherein the medical data includes data labels, and wherein the software application is configured to populate the at least one data field of the selected user interface with the received medical data by matching the data labels of the medical data to the at least one data field of the selected user interface.

9. The system of claim 1, wherein the server is configured to store the identifier of the screen that is displayed by the medical device and the medical data that is shown on the screen to a medical record associated with the patient, the medical record including an identifier of the patient, an identifier of the medical device, and medical treatment session information, and
wherein the request message includes at least one of the identifier of the patient, the identifier of the medical device, or the medical treatment session information, the request message causing the server to select the identifier of the screen that is displayed by the medical device and the medical data that is shown in the screen for transmission to the software application.

10. The system of claim 1, wherein the medical device includes at least one of a hemodialysis machine, a hemodiafiltration machine, a large-volume infusion pump, a syringe pump, a patient-controlled analgesia pump, a parenteral nutrition pump, a peritoneal dialysis machine, a continuous renal replacement therapy (“CRRT”) machine, a ventilator, a physiological monitor/sensor, or a patient bedside monitor.

11. The system of claim 1, wherein the server is a cloud-based server or provided within a medical facility.

12. The system of claim 1, wherein the medical data includes at least one of an ultrafiltration (“UF”) volume, a treatment time, a concentrate type, a sodium concentration, a bicarbonate concentration, a heparin bolus volume, a heparin flow rate, a heparin stop time, a heart rate, a dialysis fluid temperature, a dialysis fluid flow rate, dialysis fluid conductivity, UF profiling data, sodium profiling data, bicarbonate profiling data, a blood flow rate to a dialyzer, pre-and post-infusion volumes, and an indication as to whether UF is isolated, an indication whether a single needle is being used for arterial and venous connections, an event, an alarm, or diagnostic information.

13. The system of claim 1, wherein at least one of the un-populated user interfaces is an overlay user interface that is displayed over another user interface.

14. The system of claim 1, wherein the medical device is configured to:
receive physiological data from at least one sensor; and
include the physiological data with the medical data.

15. The system of claim 1, wherein the server is configured to:
receive physiological data from at least one sensor; and
include the physiological data with the medical data.

16. A system for remotely viewing medical data displayed on a screen of a medical device, the system comprising:
a user device including a processor and a memory, the memory storing instructions, which when executed by the processor, cause the processor to operate a software application, the software application including a plurality of un-populated user interfaces that correspond to a plurality of different screens of a medical device, each user interface including at least one data field corresponding to a subset of medical data that is associated with the related screen; and
a server communicatively coupled to a medical device and the user device via a network, the server configured to:
receive, from the medical device, identifiers of screens that are displayed by the medical device, the medical data that is shown on each screen in relation to a medical treatment for a patient, and timestamps when the medical data was displayed, generated, or transmitted, and
after receiving a request message from the software application of the user device, transmit the identifiers of the screens, the corresponding medical data, and the related timestamps, causing the software application to
select un-populated user interfaces that correspond to the identifiers of the screens,
populate the at least one data field of the selected user interfaces with the received medical data,
arrange the user interfaces into a sequence based on the timestamps, and
cause the user interfaces to be shown in a video-like sequential order to replay the medical treatment.

17. The system of claim 16, wherein the software application is configured to transmit the request message to the server after the medical treatment is finished.

18. The system of claim 16, wherein the server is configured to store the identifiers of the screens, the medical data, and the corresponding timestamps to a medical record including at least one of an identifier of the patient, an identifier of the medical device, and medical treatment session information, and
wherein the request message includes at least one of the identifier of the patient, the identifier of the medical device, or the medical treatment session information, the request message causing the server to select the identifiers of the screens, the medical data, and the corresponding timestamps for transmission to the software application.

19. A system for remotely viewing a screen of a medical device, the system comprising:
a medical device for performing a medical treatment for a patient, the medical device including:
a display device configured to display a medical treatment screen including medical data,
a processor configured to record an image of the medical treatment screen and compress the image, and
a digital communication module configured to receive the compressed image, decompress the image, and transmit the image to a server for storage in a memory device;
a user device including a processor and a memory, the memory storing instructions, which when executed by the processor, cause the processor to operate a software application; and
a server communicatively coupled to the medical device and the user device via a network, the server configured to:
receive the image from the digital communication module, and
after receiving a request message from the software application of the user device, transmit the image, causing the software application to display the image.

20. The system of claim 19, wherein the processor of the medical device is configured to receive a remote view input and record the image of the medical treatment screen after receiving the remote view input.

21. The system of claim 20, wherein the processor is configured to:
cause at least one of an IP address or a password to be displayed after the remote view input is received; and
transmit the at least one of the IP address or the password to the server for assessing the image,
wherein the software application is configured to display a prompt for the at least one of the IP address or the password to access the image associated with the medical device.

22. The system of claim 20, wherein the processor of the medical device is configured to associate a timestamp with the recorded image.

23. The system of claim 22, wherein the processor associates the timestamp with the recorded image by at least one of storing the timestamp as metadata for the image, displaying a watermark of the timestamp on the image, or storing the timestamp in conjunction with the image within a memory of the medical device.

24. The system of claim 22, wherein the processor is further configured to record next images of the medical treatment screen every one second to thirty seconds, associate respective timestamps with the next images, and compress the next images, wherein the digital communication module is configured to decompress the next images and transmit the next images to the server, and wherein the server transmits the next images to the software application causing the software application to order the next images based on the respective timestamps and display the ordered next images.

25. The system of claim 22, wherein the processor is further configured to record next images of the medical treatment screen when a new medical treatment screen is displayed or at least some of the medical data changes, associate respective timestamps with the next images, and compress the next images, wherein the digital communication module is configured to decompress the next images and transmit the next images to the server, and wherein the server transmits the next images to the software application causing the software application to order the next images based on the respective timestamps and display the ordered next images.

26. The system of claim 19, wherein the server is a virtual network computing (“VNC”) server and the software application is a VNC viewer application.

27. The system of claim 19, further comprising a camera configured to:
record a camera image of the medical treatment screen; and
transmit the camera image to the server,
wherein the server is configured to transmit the camera image to the software application causing the software application to display the camera image in conjunction with the image of the medical treatment screen created by the medical device.

28. The system of claim 27, wherein at least one of the server or the software application is configured to:
compare the camera image to the image of the medical treatment screen created by the medical device; and
when the camera image does not match the image of the medical treatment screen created by the medical device, provide an auditory and/or visual indication that the image of the medical treatment screen created by the medical device is not correct.

29. The system of claim 28, wherein at least one of the server or the software application is configured to perform the comparison using optical character recognition or a pixel analysis.

30. The system of claim 28, wherein the medical device is configured to show a barcode on the medical treatment screen and the camera image is configured to include the barcode, and
wherein the at least one of the server or the software application is configured to perform the comparison by comparing the barcode in the image recorded by the medical device to the barcode included within the camera image.

31. The system of claim 30, wherein the medical device is configured to periodically update the barcode.
